(19) United States
(12) Patent Application Publication
(10) Pub. No.: US 2002/0151570 A1
(43) Pub. Date: Oct. 17, 2002

(54) Thiosulfonic acid S-esters as agents for protecting material

(76) Inventors: Oliver Kretschik, Koln (DE); Martin Kugler, Leichlingen (DE); Thomas Jaetsch, Koln (DE)

Correspondence Address: BAYER CORPORATION, PATENT DEPARTMENT, 100 BAYER ROAD, PITTSBURGH, PA, 15205, US

(21) Appl. No.: 10/015,321

(22) Filed: Dec. 12, 2001

(30) Foreign Application Priority Data
Dec. 15, 2000 (DE) 10062799.4

(51) Int. Cl. A61K 31/44; A61K 31/255

(52) U.S. Cl. 514/345; 514/517

(57) ABSTRACT

The novel and known thiosulfonic acid esters of the formula (I)

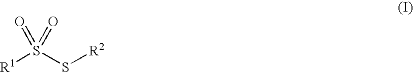

in which R¹ and R² have the meaning given in the description are highly suitable for use as biocides for protecting industrial materials. Methods for using such compositions, compositions containing such esters.

### BACKGROUND

The present invention relates to novel thiosulfonic acid esters, to processes for their preparation, to novel mixtures of thiosulfonic acid esters with other agents for protecting materials and to the use of novel and known thiosulfonic acid esters as biocides for protecting industrial materials.

Certain thiosulfonic acid esters and processes for their preparation are already known from the literature (cf., for example, Sulfur Reports, 1993, 14, 223-244; Houben-Weyl—Methoden der Organischen Chemie Vol. E 11 1985,1112-1120).

It is furthermore known that some thiosulfonic acid esters have antimicrobial action (see, for example, SU-A 198539; U.S. Pat. No. 3,346,592; Zh. Org. Khim. 1967, 3, 37; Nature 1967, 214, 4789; Khim.-Farm. Zh. 1968, 2, 12; GB 1132297; Zh. Org. Khim. 1969, 5, 62; Khim. Seraorg. Soedin., Soderzh. Neftyakh Nefteprod. 1972, 9, 282; J. Pharm. Sci. 1976, 65, 1692).

However, these known thiosulfonic acid esters have not been described as agents for protecting materials.

Surprisingly, it has been found that certain novel and known thiosulfonic acid esters are highly suitable for protecting industrial materials against attack by microorganisms.

### SUMMARY

The invention relates to a method for protecting an industrial material comprising treating the industrial material with a thiosulfonic acid ester microbiocide of the formula (I)

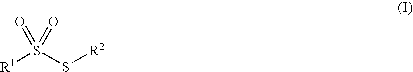

wherein R¹ and R² independently of one another represent in each case an optionally substituted alkyl, cycloalkyl, alkenyl, alkynyl, aryl or heterocyclyl; and protecting the industrial material. The invention also relates to microbiocidal compositions used in such a method, methods for making such compositions, and other methods for using such compositions. These and other features, aspects, and advantages of the present invention will become better understood with reference to the following description and appended claims.

### DESCRIPTION

The present invention provides the use of novel and known thiosulfonic acid esters of the formula (I)

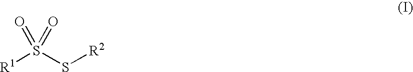

in which

R¹ and R² independently of one another represent in each case optionally substituted alkyl, cycloalkyl, alkenyl, alkynyl, aryl or heterocyclyl, as biocides for protecting industrial materials.

In the definitions of R¹ and R², the saturated or unsaturated hydrocarbon chains, such as alkyl, alkenyl or alkynyl, are in each case straight-chain or branched, including in combination with heteroatoms, such as in alkoxy or alkylthio.

Cycloalkyl represents saturated cyclic hydrocarbon radicals, such as, for example, cyclopropyl, cyclobutyl, cyclopentyl or cyclohexyl.

Aryl represents aromatic mono- or polycyclic hydrocarbon radicals, such as, for example, phenyl, naphthyl, anthranyl, phenanthranyl, preferably phenyl or naphthyl, in particular phenyl.

Heterocyclyl represents saturated and unsaturated, and also aromatic, cyclic radicals in which at least one ring member is a heteroatom, i.e. an atom differing from carbon. If the ring contains a plurality of heteroatoms, these can be identical or different. Preferred heteroatoms are oxygen, nitrogen or sulfur. If appropriate, the cyclic rings form, together with other carbocyclic or heterocyclic fused-on or bridged rings, a polycyclic ring system. A polycyclic ring system may be attached via the heterocyclic ring or via a fused-on carbocyclic ring. Preference is given to mono- or bicyclic ring systems, in particular to mono- or bicyclic aromatic ring systems.

The formula (I) provides a general definition of the novel thiosulfonic acid esters and the thiosulfonic acid esters to be used according to the invention. Preference is given to the use of compounds of the formula (I), in which

R¹ and R² independently of one another represent alkyl having 1 to 10 carbon atoms, cycloalkyl having 3 to 6 carbon atoms, alkenyl having 2 to 10 carbon atoms or alkynyl having 2 to 10 carbon atoms, which are in each case optionally mono- or polysubstituted by identical or different substituents from the group consisting of halogen; hydroxyl; alkoxy having 1 to 6 carbon atoms; halogenoalkoxy having 1 to 6 carbon atoms and 1 to 9 identical or different halogen atoms; alkylthio having 1 to 6 carbon atoms; halogenoalkylthio having 1 to 6 carbon atoms and 1 to 9 identical or different halogen atoms; acyl having 1 to 6 carbon atoms; acyloxy having 1 to 6 carbon atoms; alkoxycarbonyl having 1 to 6 carbon atoms in the alkoxy moiety; amino which is optionally mono- or disubstituted by identical or different substituents from the group consisting of C₁-C₅-alkyl and aryl; optionally substituted phenoxy; optionally substituted aryl; optionally substituted pyridyl; optionally substituted pyridyloxy; nitro; cyano, or

R¹ and R² independently of one another represent aryl which is optionally mono- to pentasubstituted by identical or different substituents from the group consisting of halogen; alkyl having 1 to 10 carbon atoms; halogenoalkyl having 1 to 8 carbon atoms and 1 to 8 identical or different halogen atoms; alkoxy having 1 to 10 carbon atoms; halogenoalkoxy having 1 to 8 carbon atoms and 1 to 8 identical or different halogen atoms; halogenoalkylthio having 1 to 8 carbon atoms and 1 to 8 identical or different halogen atoms; amino; mono- or dialkylamino having in each case straight-chain or branched alkyl radicals having in each case 1 to 6 carbon atoms; nitro, cyano, or

R¹ and R² independently of one another represent heterocyclyl which is optionally mono- to pentasubstituted by identical or different substituents from the group consisting of halogen; alkyl having 1 to 10 carbon atoms; halogenoalkyl having 1 to 8 carbon atoms and 1 to 8 identical or different halogen atoms; alkoxy having 1 to 10 carbon atoms; halogenoalkoxy having 1 to 8 carbon atoms and 1 to 8 identical or different halogen atoms; halogenoalkylthio having 1 to 8 carbon atoms and 1 to 8 identical or different halogen atoms; amino; mono- or dialkylamino having in each case straight-chain or branched alkyl radicals having in each case 1 to 6 carbon atoms; nitro; cyano.

Particular preference is given to using compounds of the formula (I), in which

R¹ and R² independently of one another represent alkyl having 1 to 8 carbon atoms, cycloalkyl having 4 to 6 carbon atoms, alkenyl having 2 to 8 carbon atoms or alkynyl having 2 to 8 carbon atoms which are in each case optionally mono- to tetrasubstituted by identical or different substituents from the group consisting of chlorine; bromine; iodine; hydroxyl; alkoxy having 1 to 5 carbon atoms; halogenoalkoxy having 1 to 5 carbon atoms and 1 to 4 identical or different chlorine, bromine or iodine atoms; alkylthio having 1 to 5 carbon atoms; halogenoalkylthio having 1 to 5 carbon atoms and 1 to 5 identical or different chlorine, bromine or iodine atoms; acyl having 1 to 5 carbon atoms; acyloxy having 1 to 5 carbon atoms; alkoxycarbonyl having 1 to 5 carbon atoms in the alkoxy moiety; amino which is optionally mono- or disubstituted by identical or different substituents from the group consisting of alkyl having 1 to 4 carbon atoms and aryl; optionally substituted phenoxy; optionally substituted aryl; optionally substituted pyridyl; optionally substituted pyridyloxy; nitro; cyano, or

R¹ and R² independently of one another represent phenyl which is optionally mono- to trisubstituted by fluorine; chlorine; alkyl having 1 to 8 carbon atoms; halogenoalkyl having 1 to 4 carbon atoms and 1 to 4 chlorine, bromine or iodine atoms; alkoxy having 1 to 8 carbon atoms; halogenoalkoxy having 1 to 4 carbon atoms and 1 to 4 chlorine, bromine or iodine atoms; halogenoalkylthio having 1 to 4 carbon atoms and 1 to 4 chlorine, bromine or iodine atoms; amino; mono- or dialkylamino having in each case straight-chain or branched alkyl radicals having in each case 1 to 5 carbon atoms; nitro; cyano, or

R¹ and R² independently of one another represent a saturated or mono- or polyunsaturated 5- or 6-membered heterocyclic ring which contains 1 to 3 heteroatoms from the group consisting of oxygen, sulfur, nitrogen and which optionally together with one or more carbocyclic or heterocyclic fused-on and/or bridged rings represents a polycyclic ring system, where the heterocyclic ring or the polycyclic ring system is optionally mono- to trisubstituted by identical or different substituents from the group consisting of fluorine; chlorine; alkyl having 1 to 8 carbon atoms; halogenoalkyl having 1 to 4 carbon atoms and 1 to 4 chlorine, bromine or iodine atoms; alkoxy having 1 to 8 carbon atoms; halogenoalkoxy having 1 to 4 carbon atoms and 1 to 4 chlorine, bromine or iodine atoms; halogenoalkylthio having 1 to 4 carbon atoms and 1 to 4 chlorine, bromine or iodine atoms; amino; monoalkylamino having straight-chain or branched alkyl radicals having 1 to 5 carbon atoms; nitro; cyano.

Very particular preference is given to the use of compounds of the formula (I), in which

R¹ and R² independently of one another represent methyl, ethyl, n- or i-propyl, n-, s-, i- or t-butyl, neo-pentyl, cyclohexyl, allyl or propargyl, which are in each case optionally mono- to trisubstituted by chlorine; hydroxyl; acyloxy having 1 to 4 carbon atoms; phenyl which is optionally mono- or disubstituted by chlorine, methyl or methoxy; nitro; cyano or represent phenyl which is optionally mono- to trisubstituted by identical or different substituents from the group consisting of fluorine; chlorine; nitro; cyano; methyl; methoxy or represent pyridyl, pyrimidyl, isoxazolyl, benzofuryl or tetrahydrofuryl.

The compounds of the formula (I), with the above-mentioned general and preferred meanings, except for the compounds:

| | |
|---|---|
| S-Methyl methanethiosulfonate | CAS No. [2949-92-0] |
| S-Ethyl ethanethiosulfonate | CAS No. [682-91-7] |
| S-(1-Methyl)ethyl 2-methyl-ethanethiocarboxylate | CAS No. [10027-69-7] |
| S-Butyl butanethiosulfonate | CAS No. [1118-40-7] |
| S-(2-Methyl)propyl 2-methyl-propanethiocarboxylate | CAS No. [59917-29-2] |
| S-(1-Methyl)propyl 1-methyl-propanethiocarboxylate | CAS No. [59917-28-1] |
| S-(2,2-Dimethyl)propyl 2,2-dimethyl-propanethio- | CAS No. [75142-07-3] |
| carboxylate | |
| S-Methyl 4-toluenethiosulfonate | CAS No. [4973-66-4] |
| S-Methyl 4-chlorobenzenethiosulfonate | CAS No. [68305-26-0] |
| S-(1-Methyl)ethyl benzenethiosulfonate | CAS No. [122217-86-1] |
| S-(1,1-Dimethyl)ethyl benzenethiosulfonate | BRG No. 7129728 |
| S-(2,2-Dimethyl)propyl benzenethiosulfonate | CAS No. [80319-02-4] |
| S-Butyl 4-toluenethiosulfonate | CAS No. [28519-31-5] |
| S-Cyclo-hexyl 4-toluenethiosulfonate | CAS No. [37556-51-7] |
| Ethyl 2-(4-chlorobenzene)-sulfonylsulfanyl-acetate | CAS No. [16599-59-0] |
| S-Cyclo-hexyl benzenethiosulfonate | CAS No. [42267-31-2] |
| Ethyl 3-benzenesulfonylsulfanyl-propionate | BRG No. 7536826 |
| Ethyl 2-benzenesulfonylsulfanyl-acetate | CAS No. [16599-55-6] |
| S-(2-Phenylcarbamoyloxy)ethyl 4- | CAS No. [4726-11-8] |
| toluenethiosulfonate | |
| S-(2-Hydroxy)ethyl 4-toluenethiosulfonate | CAS No. [125597-86-6] |
| S-4-Tolyl 4-toluenethiosulfonate | CAS No. [109163-27-1] |
| S-(4-Methoxy)phenyl 4- | CAS No. [453-43-1] |
| methoxybenzenethiosulfonate | |
| S-Methyl 2-pyridinethiosulfonate | CAS No. [22303-55-5] |
| S-(4-Cyano)phenyl 4-cyanobenzenethiosulfonate | BRG No. 3380395 |
| S-(4-Fluoro)phenyl 4-fluorobenzenethiosulfonate | CAS No. [2905-15-9] |
| S-(2-Nitro)phenyl 2-nitrobenzenethiosulfonate | CAS No. [7669-57-0]. |

are novel and also form part of the subject-matter of the present invention.

The novel compounds of the formula (I) can be prepared by reacting mercaptans of the formula (V)

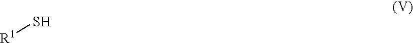

in which R¹ has the meaning given above

with sulfinic acid sodium salts of the formula (IV)

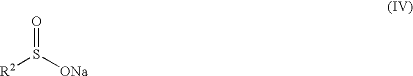

in which

R² has the meaning given above,

if appropriate in the presence of a diluent and in the presence of a halogen, such as bromine, chlorine or iodine.

Alternatively, the novel compounds of the formula (I) can be prepared by

a) oxidizing disulfides of the formula (II)

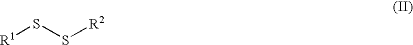

in which

R¹ and R² have the meanings given above, if appropriate in the presence of a diluent and in the presence of an oxygen-transfer agent;

b) reacting symmetrical disulfides of the formula (III)

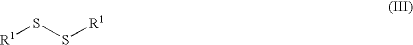

in which R¹ has the meaning given above with sulfinic acid sodium salts of the formula (IV)

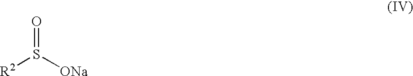

in which

R² has the meaning given above,

if appropriate in the presence of a diluent and in the presence of a halogen, such as bromine, chlorine or iodine, or

c) reacting mercaptans of the formula (V)

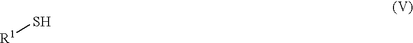

in which R¹ has the meaning given above,

if appropriate in the presence of a diluent and in the presence of sulfuryl chloride and acetic acid; or

d) reacting mercaptans of the formula (V)

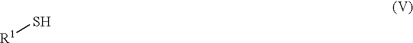

in which

R¹ has the meaning given above,

with sulfonyl chlorides of the formula (VI)

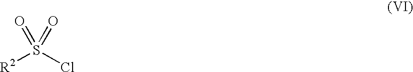

in which

R² has the meanings given above,

if appropriate in the presence of a diluent and if appropriate in the presence of a base; or

e) reacting sulfonyl chlorides of the formula (VI)

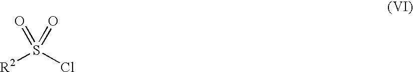

in which

R² has the meaning given above,

if appropriate in the presence of a diluent with acetyl chloride and zinc powder.

The novel and known compounds of the formula (I) have potent microbicidal action and can be used for controlling undesirable microorganisms, such as fungi and bacteria, in the protection of materials.

In the protection of materials, the substances according to the invention can be used for protecting industrial materials against attack and destruction by undesirable microorganisms.

In the present context, industrial materials are to be understood as meaning non-live materials which have been prepared for use in industry. For example, industrial materials can be glues, sizes, paper and board, textiles, leather, wood, wooden materials, paints and synthetic articles, cooling lubricants and other materials which can be attacked or destroyed by microorganisms. Parts of production plants, for example cooling-water circuits, which may be impaired by the multiplication of microorganisms may also be mentioned as industrial materials in the context of the present invention. Industrial materials which are preferably to be protected are adhesives, sizes, paper and boards, leather, wood, paints, cooling lubricants and heat transfer liquids.

Examples of microorganisms which are capable of bringing about degradation of, or change in, the industrial materials and which may be mentioned are bacteria, fungi, yeasts, algae and slime organisms. The active compounds according to the invention preferably act against fungi, in particular moulds, wood-discoloring and wood-destroying fungi (Basidiomycetes) and also against slime organisms and algae. Microorganisms of the following genera may be mentioned by way of example:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa,

Staphylococcus, such as Staphylococcus aureus.

Depending on their particular physical and/or chemical properties, the active compounds can be converted to the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is, liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Suitable liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as highly disperse silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates. Suitable dispersants are: for example lignin-sulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention, as such or in their formulations, can also be used in a mixture with known fungicides, bactericides, acaricides, nematicides or insecticides, for example to widen the activity spectrum or to prevent the development of resistance. In many cases, synergistic effects are obtained, i.e. the activity of the mixture is greater than the activity of the individual components.

For applications in the protection of materials, the following co-components, for example, are found to be particularly favorable:

imidazoles such as: clotrimazole, bifonazole, climbazole, econazole, fenapamil, imazalil, isoconazole, ketoconazole, lombazole, miconazole, pefurazoate, prochloraz, triflumizole, thiazolcar, 1-imidazolyl-1-(4′-chlorophenoxy)-3,3-dimethylbutan-2-one, and their metal salts and acid adducts;

triazoles such as: azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, metconazole, myclobutanil, paclobutrazol, penconazole, propioconazole, (±)-cis-1-(4-chlorophenyl)-2-(1H-1,2 ,4-triazol-1-yl)-cycloheptanol, 2-(1-tert-butyl)-1-(2-chlorophenyl)-3-(1,2,4-triazol-1-yl)-propan-2-ol, tebuconazole, tetraconazole, triadimefon, triadimenol, triapenthenol, triflumizole, triticonazole, uniconazole and their metal salts and acid adducts;

pyridines and pyrimidines such as: ancymidol, buthiobate, fenarimol, mepanipyrin, nuarimol, pyvoxyfur, triamirol;

succinate dehydrogenase inhibitors such as: benodanil, carboxim, carboxim sulfoxide, cyclafluramid, fenfuram, flutanil, furcarbanil, furmecyclox, mebenil, mepronil, methfuroxam, metsulfovax, pyrocarbolid, oxycarboxin, shirlan, Seedvax;

naphthalene derivatives such as: terbinafine, naftifine, butenafine, 3-chloro-7-(2-aza-2,7,7-trimethyl-oct-3-en-5-ine);

sulfenamides such as: dichlofluanid, tolylfluanid, folpet, fluorofolpet, captan, captofol;

benzimidazoles such as: carbendazim, benomyl, fuberidazole, thiabendazole or their salts;

morpholine derivatives such as: aldimorph, dimethomorph, dodemorph, falimorph, fenpropidin fenpropimorph, tridemorph, trimorphamid and their arylsulfonate salts such as, for example, p-toluenesulfonic acid and p-dodecylphenyl-sulfonic acid;

benzothiazoles such as: 2-mercaptobenzothiazole;

benzothiophene dioxides such as: N-cyclohexyl-benzo[b]thiophene-S,S-dioxide carboxamide;

benzamides such as: 2,6-dichloro-N-(4-trifluoromethylbenzyl)-benzamide, tecloftalam;

boron compounds such as: boric acid, boric ester, borax;

formaldehyde and formaldehyde-releasing compounds such as: benzyl alcohol mono-(poly)-hemiformal, n-butanol hemiformal, dazomet, ethylene glycol hemiformal, hexa-hydro-S-triazine, hexamethylenetetramine, N-hydroxymethyl-N′-methylthiourea, N-methylolchloroacetamide, oxazolidine, paraformaldehyde, taurolin, tetrahydro-1,3-oxazine, N-(2-hydroxypropyl)-amine-methanol;

isothiazolinones such as: N-methylisothiazolin-3-one, 5-chloro-N-methylisothiazolin-3-one, 4,5-dichloro-N-octylisothiazolin-3-one, 5-chloro-N-octylisothiazolinone, N-octyl-isothiazolin-3-one, 4,5-trimethylene-isothiazolinone, 4,5-benzoisothiazolinone;

aldehydes such as: cinnamaldehyde, formaldehyde, glutardialdehyde, β-bromocinnamaldehyde;

thiocyanates such as: thiocyanatomethylthiobenzothiazole, methylenebisthiocyanate;

quaternary ammonium compounds and guanidines such as: benzalkonium chloride, benzyldimethyltetradecylammonium chloride, benzyldimethyldodecylammonium chloride, dichlorobenzyl-dimethyl-alkyl-ammonium chloride, didecyldimethylammonium chloride, dioctyl-dimethyl-ammonium chloride, N-hexadecyl-trimethyl-ammonium chloride, 1-hexadecyl-pyridinium chloride, iminoctadine tris(albesilate);

iodine derivatives such as: diiodomethyl p-tolyl sulfone, 3-iodo-2-propinyl alcohol, 4-chlorophenyl-3-iodopropargylformal, 3-bromo-2,3-diiodo-2-propenyl ethylcarbamate, 2,3,3-triiodoallyl alcohol, 3-bromo-2,3-diiodo-2-propenyl alcohol, 3-iodo-2-propinyl n-butyl-carbamate, 3-iodo-2-propinyl n-hexylcarbamate, 3-iodo-2-propinyl-cyclohexylcarbamate, 3-iodo-2-propinyl phenylcarbamate;

phenols such as: tribromophenol, tetrachlorophenol, 3-methyl-4-chlorophenol, 3,5-dimethyl-4-chlorophenol, phenoxyethanol, dichlorophene, 2-benzyl-4-chlorophenol, 5-chloro-2-(2,4-dichlorophenoxy)-phenol, hexachlorophene, p-hydroxybenzoate, o-phenylphenol, m-phenylphenol, p-phenylphenol and their alkali metal salts and alkaline earth metal salts;

microbicides with an activated halogen group such as: bronopol, bronidox, 2-bromo-2-nitro-1,3-propanediol, 2-bromo-4′-hydroxy-acetophenone, 1-bromo-3-chloro-4,4,5,5-tetramethyl-2-imidazolidinone, β-bromo-β-nitrostyrene, chloracetamid, chloramin T, 1,3-dibromo-4,4,5,5-tetramethyl-2-imidazolidinone, dichloramin T, 3,4-dichloro-(3H)-1,2-dithiol-3-one, 2,2-dibromo-3-nitrile-propionamide, 1,2-dibromo-2,4-dicyanobutane, halane, halazone, mucochloric acid, phenyl (2-chlorocyano-vinyl) sulfone, phenyl (1,2-dichloro-2-cyanovinyl) sulfone, trichloroisocyanuric acid;

pyridines such as: 1-hydroxy-2-pyridinethione (and their Na, Fe, Mn, Zn salts), tetrachloro-4-methylsulfonylpyridine, pyrimethanol, mepanipyrim, dipyrithion, 1-hydroxy-4-methyl-6-(2,4,4-trimethylpentyl)-2(1H)-pyridine;

methoxyacrylates or similar such as: azoxystrobin, methyl (E)-methoximino[alpha-(o-tolyloxy)-o-tolyl]acetate, (E)-2-methoxyimino-N-methyl-2-(2-phenoxyphenyl)acetamide, (E)-2-{2-[6-(2-cyanophenoxy)pyrimidin-4-yloxy]phenyl}-3-methoxyacrylate, 0-methyl 2-[([3-methoximino-2-butyl)imino]oxy)o-tolyl]-2-methoximino-acet-imidate, 2-[[[[1-(2,5-dimethylphenyl)ethylidene]amino]oxy]methyl]-alpha-(methoximino)-N-methylbenzeneacetamide, alpha-(methoxyimino)-N-methyl-2-[[[[1-[3-(trifluoromethyl)phenyl]ethylidene]amino]oxy]methyl]-benzeneacetamide, trifloxystrobin, alpha-(rnethoxymethylene)-2-[[[[1-[3-(trifluoromethyl)phenyl]ethylidene]amino]oxy]methyl]-benzeneacetic acid methyl ester, 2-[[[5-chloro-3-(trifluoromethyl)-2-pyridinyl]oxy]methyl]-alpha-(methoxyimino)-N-methylbenzeneacetamide, 2-[[[cyclopropyl[(4-ethoxyphenyl)imino]methyl]thio]methyl]-alpha-(methoxyimino)-benzeneacetic acid methyl ester, alpha-(methoxyimino)-N-methyl-2-(4-methyl-5-phenyl-2,7-dioxa-3 ,6-diazaocta-3,5-d ien-1-yl)-benzeneacetamide, alpha-(methoxymethylene)-2-(4-methyl-5-phenyl-2,7-dioxa-3,6-diazaocta-3,5-dien-1-yl)-benzeneacetic acid methyl ester, alpha-(methoxyimino)-N-methyl-2-[[[1-[3-(trifluoromethyl)phenyl]ethoxy]imino]-methyl]-benzeneacetamide, 2-[[(3,5-dichloro-2-pyridinyl)oxy]methyl]-alpha-(methoxyim ino)-N-methyl-benzeneacetam ide, 2-[4,5-dimethyl-9-(4-morpholinyl)-2,7-dioxa-3,6-diazanona-3,5-dien-1-yl]-alpha-(methoxymethylene)-benzeneacetic acid methyl ester, kresoxim-methyl;

metal soaps such as: tin naphthenate, copper napthenate, zinc napthenate, tin octoate, copper octoate, zinc octoate, tin 2-ethylhexanoate, copper 2-ethylhexanoate, zinc 2-ethylhexanoate, tin oleate, copper oleate, zinc oleate, tin phosphate, copper phosphate, zinc phosphate, tin benzoate, copper benzoate, zinc benzoate;

metal salts such as: copper hydroxycarbonate, sodium dichromate, potassium dichromate, potassium chromate, copper sulfate, copper chloride, copper borate, zinc fluorosilicate, copper fluorosilicate;

oxides such as: tributyltin oxide, Cu₂O, CuO, ZnO;

dithiocarbamates such as: cufraneb, ferban, potassium N-hydroxymethyl-N′-methyl-dithiocarbamate, sodium dimethyidithiocarbamate, potassium dimethyldithiocarbamate, macozeb, maneb, metam, metiram, thiram, zineb, ziram;

nitriles such as: 2,4,5,6-tetrachloroisophthalonitrile, disodium cyanodithioimidocarbamate;

quinolines such as: 8-hydroxyquinoline and its copper salts;

other fungicides and bactericides such as: 5-hydroxy-2(5H)-furanone, 4,5-benzodithiazolinone, 4,5-trimethylenedithiazolinone, N-(2-p-chlorobenzoylethyl)-hexaminium chloride, 2-oxo-2-(4-hydroxy-phenyl)-acetohydroxamic acid chloride, tris-N-(cyclohexyldiazeniumdioxy)-aluminum, N-(cyclohexyldiazeniumdioxy)-tributyltin or its potassium salts, bis-N-(cyclohexyldiazeniumdioxy)-copper, iprovalicarb, fenhexamid, spiroxamine, carpropamid, diflumetorin, quinoxyfen, famoxadone, polyoxorim, acibenzolar S-methyl, furametpyr, thifluzamide, methalaxyl-M, Ag-, Zn- or Cu-containing zeolites alone or incorporated into polymeric materials.

Very especially preferred are mixtures of compounds of the formula (I) to be used according to the invention with-one or more of the following active compounds: azaconazole, bromuconazole, cyproconazole, dichlobutrazol, diniconazole, hexaconazole, metaconazole, penconazole, propiconazole, tebuconazole, dichlofluanid, tolylfluanid, fluorfolpet, methfuroxam, carboxin, benzo[b]thiophene S,S-dioxide N-cyclohexyl-carboxamide, fenpiclonil, 4-(2,2-difluoro-1,3-benzodioxol-4-yl)-1H-pyrrole-3-carbonitrile, butenafine, imazalil, N-methyl-isothiazolin-3-one, 5-chloro-N-methylisothiazolin-3-one, N-octylisothiazolin-3-one, dichloro-N-octylisothiazolinone, mercaptobenthiazole, thiocyanatomethyl-thiobenzothiazole, benzoisothiazolinone, N-(2-hydroxypropyl)-amino-methanol, benzyl alcohol (hemi)-formal, N-methylolchloroacetamide, N-(2-hydroxypropyl)-amine-methanol, glutaraldehyde, omadine, dimethyl dicarbonate, 2-bromo-2-nitro-1,3-propanediol and/or 3-iodo-2-propinyl n-butylcarbamate.

Apart from with the above-mentioned fungicides and bactericides, mixtures with a good efficacy are, moreover, also prepared, for example with one or more of the following active compounds:

insecticides/acaricides/nematicides: abamectin, acephate, acetamiprid, acrinathrin, alanycarb, aldicarb, aldoxycarb, aldrin, allethrin, alpha-cypermethrin, amitraz, avermectin, AZ 60541, azadirachtin, azinphos A, azinphos M, azocyclotin, Bacillus thuringiensis, barthrin, 4-bromo-2(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, bioresmethrin, bioallethrin, bromophos A, bromophos M, bufencarb, buprofezin, butathiophos, butocarboxim, butoxycarboxim, cadusafos, carbaryl, carbofuran, carbophenothion, carbosulfan, cartap, quinomethionate, cloethocarb, chlordane, chlorethoxyfos, chlorfenapyr, chlorfenvinphos, chlorfluazuron, chlormephos, N-[(6-chloro-3-pyridinyl)-methyl]-N′-cyano-N-methyl-ethaneimidamide, chlorpicrin, chlorpyrifos A, chlorpyrifos M, cis-resmethrin, clocythrin, cypophenothrin clofentezin, coumaphos, cyanophos, cycloprothrin, cyfluthrin, cyhalothrin, cyhexatin, cypermethrin, cyromazin, decamethrin, deltamethrin, demeton M, demeton S, demeton-S-methyl, diafenthiuron, dialiphos, diazinon, 1,2-dibenzoyl-1(1,1-dimethyl)-hydrazine, DNOC, dichlofenthion, dichlorvos, dicliphos, dicrotophos, difethialone, diflubenzuron, dimethoate, dimethyl-(phenyl)-silyl-methyl 3-phenoxybenzyl ether, dimethyl-(4-ethoxyphenyl)-silylmethyl-3-phenoxybenzyl ether, dimethylvinphos, dioxathion, disulfoton, eflusilanate, emamectin, empenthrin, endosulfan, EPN, esfenvalerate, ethiofencarb, ethion, ethofenprox, etrimphos, etoxazole, etobenzanid, fenamiphos, fenazaquin, fenbutatin oxide, fenfluthrin, fenitrothion, fenobucarb, fenothiocarb, fenoxycarb, fenpropathrin, fenpyrad, fenpyroximat, fensulfothion, fenthion, fenvalerate, fipronil, fluazuron, flucycloxuron, flucythrinate, flufenoxuron, flupyrazofos, flufenzine, flumethrin, flufenprox, fluvalinate, fonophos, formethanate, formothion, fosmethilan, fosthiazate, fubfenprox, furathiocarb, halofenocid, HCH, heptenophos, hexaflumuron, hexythiazox, hydramethylnon, hydroprene, imidacloprid, imiprothrin, indoxycarb, iodfenfos, iprinomectin, iprobenfos, isazophos, isoamidophos, isofenphos, isoprocarb, isoprothiolane, isoxathion, ivermectin, iama-cyhalothrin, lufenuron, kadedrin lambda-cyhalothrin, lufenuron, malathion, mecarbam, mervinphos, mesulfenphos, metaldehyde, methacrifos, methamidophos, methidathion, methiocarb, methomyl, metalcarb, milbemectin, monocrotophos, moxiectin, naled, NC 184, NI 125, nicotine, nitenpyram, omethoate, oxamyl, oxydemethon M, oxydeprofos, parathion A, parathion M, penfluron, permethrin, 2-(4-phenoxyphenoxy)-ethyl ethylcarbamate, phenthoate, phorate, phosalon, phosmet, phosphamidon, phoxim, pirimicarb, pirimiphos M, pirimiphos A, prallethrin, profenophos, promecarb, propaphos, propoxur, prothiophos, prothoate, pymetrozin, pyrachlophos, pyridaphenthion, pyresmethrin, pyrethrum, pyridaben, pyrimidifen, pyriproxifen, pyrithiobac-sodium, quinalphos, resmethrin, RH-7988, rotenone, salithion, sebufos, silafluofen, spinosad, sulfotep, sulprofos, tau-fluvalinate, taroils, tebufenozide, tebufenpyrad, tebupirimphos, teflubenzuron, tefluthrin, temephos, terbam, terbufos, tetrachlorvinphos, tetramethrin, tetramethacarb, thiacloprid, thiafenox, thiamethoxam, thiapronil, thiodicarb, thiofanox, thiazophos, thiocyclam, thiomethon, thionazin, thuringiensin, tralomethrin, transfluthrin, triarathen, triazophos, triazamate, triazuron, trichlorfon, triflumuron, trimethacarb, vamidothion, XMC, xylylcarb, zetamethrin; molluscicides fentin acetate, metaldehyde, methiocarb, niclosamide; herbicides and algicides acetochlor, acifluorfen, aclonifen, acrolein, alachlor, alloxydim, ametryn, amidosulfuron, amitrole, ammonium sulfamate, anilofos, asulam, atrazine, azafenidin, aziptrotryne, azimsulfuron, benazolin, benfluralin, benfuresate, bensulfuron, bensulfide, bentazone, benzofencap, benzthiazuron, bifenox, bispyribac, borax, bromacil, bromobutide, bromofenoxim, bromoxynil, butachlor, butamifos, butralin, butylate, bialaphos, benzoyl-prop, bromobutide, butroxydim, carbetamide, carfentrazone-ethyl, carfenstrole, chlomethoxyfen, chloramben, chlorbromuron, chlorflurenol, chloridazon, chlorimuron, chlornitrofen, chloroacetic acid, chloransulam-methyl, cinidon-ethyl, chlorotoluron, chloroxuron, chlorpropham, chlorsulfuron, chlorthal, chlorthiamid, cinmethylin, cinofulsuron, clefoxydim, clethodim, clomazone, chlomeprop, clopyralid, cyanamide, cyanazine, cycloate, cycloxydim, chloroxynil, clodinafop-propargyl, cumyluron, CGA 248757, clometoxyfen, cyhalofop, cyhalofop-butyl, clopyrasuluron, cyclosulfamuron, diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipropetryn, diquat, dithiopyr, diduron, DNOC, DSMA, 2,4-D, daimuron, dalapon, dazomet, 2,4-DB, desmedipham, desmetryn, dicamba, dichlobenil, dimethamid, dithiopyr, dimethametryn,eglinazine, endothal, EPTC, esprocarb, ethalfluralin, ethidimuron, ethofumesate, ethobenzanid, ethoxyfen, ET 751, ethametsulfuron, ethoxysulfuron, fenoxaprop, fenoxaprop-P, fenuron, flamprop, flamprop-M, fiazasulfuron, fluazifop, fluazifop-P, fuenachlor, fluchloralin, flufenacet, flumeturon, fluorocglycofen, fluoronitrofen, flupropanate, flurenol, fluridone, flurochloridone, fluroxypyr, fomesafen, fosamine, flamprop-isopropyl, flamprop-isopropyl-L, flumiclorac-pentyl, flumipropyn, flumioxzim, flurtamone, flumioxzim, flupyrsulfuron-methyl, glyphosate, glufosinate-ammonium haloxyfop, hexazinone, imazamethabenz, isoproturon, isoxaben, isoxapyrifop, imazapyr, imazaquin, imazethapyr, ioxynil, isopropalin, imazosulfuron, imazomox, isoxaflutole, imazapic, lactofen, lenacil, linuron, LS830556, MCPA, MCPA-thioethyl, MCPB, mecoprop, mecoprop-P, mefenacet, mefluidide, metam, metamitron, metazachlor, methabenzthiazuron, methazole, methoroptryne, methyldymron, methyl isothiocyanate, metobromuron, metoxuron, metribuzin, metsulfuron, molinate, monalide, monolinuron, MSMA, metolachlor, metosulam, metobenzuron, naproanilide, napropamide, naptalam, neburon, nicosulfuron, norflurazon, sodium chlorate, oxadiazon, oxyfluorfen, oxysulfuron, orbencarb, oryzalin, oxadiargyl, propyzamide, prosulfocarb, pyrazolate, pyrazolsulfuron, pyrazoxyfen, pyribenzoxim, pyributicarb, pyridate, paraquat, pebulate, pendimethalin, pentachlorophenol, pentoxazone, pentanochlor, petroleum oils, phenmedipham, picloram, piperophos, pretilachlor, primisulfuron, prodiamine, prometryn, propachlor, propanil, propaquizafob, propazine, propham, propisochlor, pyriminobac-methyl, pelargonic acid, pyrithiobac, quinmerac, quinocloamine, quizalofop, quizalofop-P, quinchlorac, rimsulfuron sethoxydim, sifuron, simazine, simetryn, sulfosulfuron, sulfometuron, sulfentrazone, sulcotrione, sulfosate, tar oils, TCA, tebutam, tebuthiuron, terbacil, terbumeton, terbuthylazine, terbutryn, thiazafluoron, thifensulfuron, thiobencarb, thiocarbazil, tralkoxydim, tri-allate, triasulfuron, tribenuron, triclopyr, tridiphane, trietazine, trifluoralin, tycor, thdiazimin, thiazopyr, triflusulfuron, vernolate.

The active compounds can be applied as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading-on and the like.

The compositions used for protecting industrial materials generally comprise the active compounds in an amount of from 1 to 95%, preferably from 10 to 75%.

The use concentrations of the active compounds according to the invention depend on the type and the occurrence of the microorganisms to be controlled, and on the composition of the material to be protected. The optimal rate can be determined by test series. In general, the use concentrations are in the range from 0.001 to 5% by weight, preferably from 0.05 to 1.0% by weight, based on the material to be protected.

The invention is further described in the following illustrative examples in which all parts and percentages are by weight unless otherwise indicated.

### PREPARATION EXAMPLES

### Example 1

At from 0to −5° C., 23.06 g (171 mmol) of sulfuryl chloride were added dropwise to a solution of 10.87 g (110 mmol) of isobutylthiol and 3.33 g (56 mmol) of acetic acid in 30 ml of dichloromethane, and the mixture was stirred for 12 h. The mixture was washed three times with water and the organic phase was dried and concentrated using a rotary evaporator. Column-chromatographic purification (SiO₂, toluene/hexane=1/1) of the residue gives the thiosulfonic acid ester of the formula (I) where R¹ and R²=iso-C₄H₉- as a colorless oil.

Yield: 8.11 g (70% of theory), n_{D}²³=1.4833.

### Example 2

At 0° C., 0.44 g (1.8 mmol) of 3-chloro-perbenzoic acid was added a little at a time to a solution of 0.50 g (0.9 mmol) of 2-({2-[(2,2-dicyclohexylacetyl)oxy]ethyl}-disulfanyl)ethyl dicyclohexylacetate in 25 ml of trichloromethane, and the mixture was stirred at room temperature for 5 h. The mixture was concentrated using a rotary evaporator and the residue was purified by column chromatography (SiO₂, toluene). This gives the thiosulfonic acid ester of the formula (I) in which R¹ and R² represent

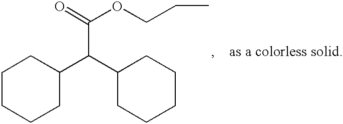

Yield: 0.23 g (44% of theory), m.p.: 85° C.

### Example 3

1.45 g (16 mmol) of 1-mercapto-2-propanol and 4.00 g (16 mmol) of (2-methoxy-5-nitrophenyl)methanesulfinic acid sodium salt were suspended in 75 ml of dichloromethane and treated dropwise with a solution of 1.26 g (79 mmol) of bromine in 15 ml of dichloromethane. After the addition has ended, the mixture was stirred for another 6 h, the remaining solid was filtered off with suction and the filtrate was concentrated using a rotary evaporator. The residue gives, after work-up by column chromatography (SiO₂, toluene/ethyl acetate=1/1), the thiosulfonic acid ester of the formula (I) where

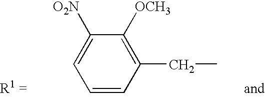

R²=CH₃—CHOH—CH₂—as a colorless oil.

Yield: 0.85 g (17% of theory), n_{D}²⁴=1.5924.

### Example 4

4.58 g (30 mmol) of diisopropyldisulfide and 10.00 g (60 mmol) of benzenesulfinic acid sodium salt were suspended in 100 ml of dichloromethane and treated dropwise with a solution of 4.88 g (30 mmol) of bromine in 15 ml of dichloromethane. After the addition has ended, the mixture was stirred for another 6 h, the remaining solid was filtered off with suction and the filtrate was concentrated using a rotary evaporator. Without further purification, the thiosulfonic acid ester (I) where

R¹=C₆H₅— and

R²=iso-C₃H₇—was obtained as a colorless oil.

Yield: 12.76 g (97% of theory), n_{D}²³=1.5561.

### Example 5

At 0° C., a solution of 5.00 g of p-toluenesulfonyl chloride in 15 ml of dichloromethane was added dropwise to a solution of 2.78 g (26 mmol) of 1-mercapto-2-butanol and 2.65 g (26 mmol) of triethylamine in 100 ml of dichloromethane, and the mixture was stirred at room temperature for another 6 h. The mixture was washed three times with saturated sodium carbonate solution, the organic phase was dried and concentrated using a rotary evaporator and the residue was purified by column chromatography (SiO₂, toluene). This gives the thiosulfonic acid ester (I) where

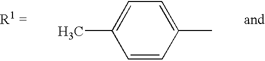

R²=CH₃—CH₂—CHOH-CH₂—-as a colorless oil.

Yield: 0.62 g (9% of theory), n_{D}²⁶=1.5233.

### Example 6

3.00 g (46 mmol) of zinc dust were suspended in 150 ml of ethyl acetate, and to activate the zinc, the mixture was then heated at reflux with a few drops of dibromomethane and trimethylsilyl chloride for 60 min. After cooling to room temperature, 5.48 g (29 mmol) of p-toluenesulfonyl chloride were added, and 2.25 g (29 mmol) of acetyl chloride were then added dropwise with cooling, the temperature being kept below 40° C. The mixture was stirred at room temperature for 3 h and then washed with 1 N HCl solution and saturated NaCl solution. The organic phase was dried over sodium sulfate and concentrated using a rotary evaporator. Crystallization of the residue from hexane gives the thiosulfonic acid ester (I) where

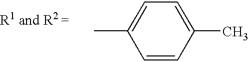

as a colorless solid.

Yield: 2.72 g (34% of theory), m.p.: 73° C.

The compounds (I) listed in Table 1 were prepared analogously to Examples 1 to 6 and/or in accordance with the general statements in the description of the experiments.

TABLE 1

| | |
|---|---|
| | (I) |
| 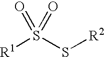 | |

| Example | R¹ | R² | Physical data |
|---|---|---|---|
| 7 | H₃C— | —CH₃ | n_{D}²⁰ = 1.5130 |
| 8 | H₅C₂— | —C₂H₅ | n_{D}²³ = 1.4933 |
| 9 | iso-C₃H₇— | -iso-C₃H₇ | n_{D}²³ = 1.4897 |
| 10 | n-C₄H₉— | -n-C₄H₉ | n_{D}²² = 1.4883 |
| 11 | sec-C₄H₉— | -sec-C₄H₉ | n_{D}²³ = 1.4905 |
| 12 | neo-C₅H₁₁— | -neo-C₅H₁₁ | m.p. = 60° C. |
| 13 | (2-OCH₃-3-NO₂—C₆H₄)—CH₂— | —CH₂—(C₆H₄-4-Cl) | m.p. = 115° C. |
| 14 | (2-OCH₃-3-NO₂—C₆H₄)—CH₂— | -sec-C₄H₉ | m.p. = 93° C. |
| 15 | (2-OCH₃-3-NO₂—C₆H₄)—CH₂— | —CH₃ | m.p. = 119° C. |
| 16 | (2-OCH₃-3-NO₂—C₆H₄)—CH₂— | —C₂H₅ | m.p. = 95° C. |
| 17 | (2-OCH₃-3-NO₂—C₆H₄)—CH₂— | -cyclo-C₆H₁₁ | m.p. = 99° C. |
| 18 | (2-OCH₃-3-NO₂—C₆H₄)—CH₂— | -n-C₄H₉ | m.p. = 88° C. |
| 19 | (2-OCH₃-3-NO₂—C₆H₄)—CH₂— | -iso-C₃H₇ | m.p. = 95° C. |
| 20 | (2-OCH₃-3-NO₂—C₆H₄)—CH₂— | —CH₂CH₂CO₂C₂H₅ | m.p. = 116° C. |
| 21 | (2-OCH₃-3-NO₂—C₆H₄)—CH₂— | —CH₂CH₂—OH | m.p. = 75° C. |
| 22 | (2-OCH₃-3-NO₂—C₆H₄)—CH₂— | -iso-C₄H₉ | m.p. = 56° C. |
| 23 | 4-CH₃—C₆H₄— | —CH₃ | m.p. = 45° C. |
| 24 | 4-Cl—C₆H₄— | —CH₃ | n_{D}²⁶ = 1.5665 |
| 25 | 4-OCH₃—C₆H₄— | —CH₃ | n_{D}²³ = 1.5443 |
| 26 | C₆H₅— | -sec-C₄H₉ | n_{D}²³ = 1.5494 |
| 27 | C₆H₅— | -tert-C₄H₉ | n_{D}²³ = 1.6374 |
| 28 | C₆H₅— | -neo-C₅H₁₁ | n_{D}²⁴ = 1.5436 |
| 29 | C₆H₅— | —CH₂-(3-Cl-4-Cl—C₆H₄) | m.p. = 53° C. |
| 30 | 4-CH₃—C₆H₄— | -n-C₄H₉ | n_{D}²⁴ = 1.5519 |
| 31 | 4-Cl—C₆H₄— | -sec-C₄H₉ | n_{D}²⁴ = 1.5645 |
| 32 | 4-F—C₆H₄— | -iso-C₃H₇ | n_{D}²⁴ = 1.5384 |
| 33 | (4-Cl-3-NO₂—C₆H₄)— | -tert-C₄H₉ | n_{D}²⁴ = 1.6064 |
| 34 | (4-CH₃-3-NO₂—C₆H₄)— | -n-C₄H₉ | n_{D}²⁴ = 1.5684 |
| 35 | 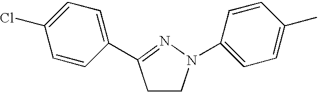 | -sec-C₄H₉ | m.p. = 165° C. |
| 36 | 4-CH₃—C₆H₄— | -cyclo-C₆H₁₁ | m.p. = 52° C. |
| 37 | 4-Cl—C₆H₄— | —CH₂CO₂C₂H₅ | n_{D}²⁴ = 1.5675 |
| 38 | C₆H₅— | -cyclo-C₆H₁₁ | ¹H NMR(CDCl₃): |
| | | | δ = 1.2-2.0, m, 10H; |
| 39 | C₆H₅— | —CH₂CH₂CO₂C₂H₅ | ¹H NMR(CDCl₃): δ = |
| | | | 1.3, t, 3H: 3.6, s, 2H: |
| 40 | C₆H₅— | —CH₂CO₂C₂H₅ | ¹H NMR(CDCl₃): δ = |
| | | | 1.3, t, 3H; 3.6, s, 2H; |
| 41 | C₆H₅— | 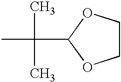 | ¹H NMR (CDCl₃): δ =1.35, s, 6H; 3.8-4.0. |
| 42 | 4-CH₃—C₆H₄— | —CH₂CH₂O—CO—NH—C₆H₅ | m.p. = 60° C. |
| 43 | 4-Cl—C₆H₄— | -cyclo-C₆H₁₁ | ¹H NMR(CDCl₃): δ = |
| | | | δ = 1.2-2.0, m, 10H; |
| 44 | 4-Cl—C₆H₄— | —CH₂CH₂CO₂C₂H₅ | ¹H NMR(CDCl₃): δ = |
| | | | 1.2, t, 3H: 2.7, d, 2H; |
| 45 | 4-Cl—C₆H₄— | —CH₂—(C₆H₄-4-Cl) | m.p. = 66° C. |
| 46 | 4-F—C₆H₄— | -cyclo-C₆H₁₁ | ¹H NMR(CDCl₃): |
| | | | δ = 1.2-2.0, m, 10H; |
| 47 | 4-F—C₆H₄— | —CH₂CH₂CO₂C₂H₅ | m.p. = 45° C. |
| 48 | 4-F—C₆H₄— | —CH₂CH₂O—CO—NH—C₆H₅ | m.p. = 75° C. |
| 49 | 4-F—C₆H₄— | —CH₂CH₂OH | N_{D}²⁶ = 1.5666 |
| 50 | 4-C≡N—C₆H₄— | —CH₂CH₂OH | N_{D}²⁶ = 1.6046 |
| 51 | 4-Cl—C₆H₄— | —CH₂CH₂OH | N_{D}²⁶ = 1.6005 |
| 52 | 4-CH₃—C₆H₄— | —CH₂CH₂OH | N_{D}²⁶ = 1.5810 |
| 53 | 4-C≡N—C₆H₄— | -cyclo-C₆H₁₁ | m.p. = 51° C. |
| 54 | 4-C≡N—C₆H₄— | —CH₂CH₂O—CO—NH—C₆H₅ | m.p. = 120° C. |
| 55 | 4-C≡N—C₆H₄— | —CH₂CH₂CO₂C₂H₅ | m.p. = 66° C. |
| 56 | 4-C≡N—C₆H₄— | —CH₂—C₆H₅ | m.p. = 68° C. |
| 57 | 4-C≡N—C₆H₄— | —CH₂—(C₆H₅-4-Cl) | m.p. = 96° C. |
| 58 | 4-CH₃—C₆H₄— | —CH₂—CHOH—CH₃ | n_{D}²⁶ = 1.5709 |
| 59 | 4-CH₃—C₆H₄— | —CH₂—CO—(C₆H₄-4-Cl) | m.p. = 179° C. |
| 60 | 4-Cl—C₆H₄— | —CH₂—CO—(C₆H₄-4-Cl) | m.p. = 175° C. |
| 61 | 4-CH₃—C₆H₄— | 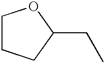 | m.p. = 42° C. |
| 62 | 4-CH₃—C₆H₄— | —CH₂—CHOH—CH₂OH | n_{D}²⁴ = 1.5827 |
| 63 | 4-CH₃—C₆H₄— | 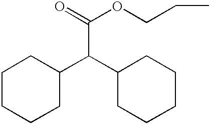 | n_{D}²⁴ = 1.5436 |
| 64 | 4-CH₃—C₆H₄— | —CH₂—CHCH₃—O₂C—C₆H₅ | n_{D}²⁴ = 1.5789 |
| 65 | 4-Cl—C₆H₄— | —CH₂—CHOH—CH₃ | n_{D}²⁴ = 1.5815 |
| 66 | 4-F—C₆H₄— | —CH₂—CHOH—CH₃ | n_{D}²⁴ = 1.5558 |
| 67 | 4-Cl—C₆H₄— | —CH₂—CHOH—CH₂OH | m.p. = 77° C. |
| 68 | 4-F—C₆H₄— | —CH₂—CHOH—CH₂OH | ¹H NMR(CDCl₃): δ = |
| | | | 1.9, br, 2OH: 3.1, m. |
| 69 | 4-CH₃—C₆H₄— | —CH₂—CHCH₃—O₂CCH₃ | n_{D}²³ = 1.5329 |
| 70 | (3-NO₂-4-CH₃—C₆H₄)— | —CH₂—CHOH—CH₃ | n_{D}²³ = 1.5880 |
| 71 | (3-NO₂-4-CH₃—C₆H₄)— | —CH₂—CHOH—CH₂OH | m.p. = 52° C. |
| 72 | 2-NO₂—C₆H₄— | —CH₂—CHOH—CH₃ | n_{D}²² = 1.5347 |
| 73 | (3-NO₂-4-CH₃—C₆H₄)— | -cyclo-C₆H₁₁ | n_{D}²³ = 1.5778 |
| 74 | (3-NO₂-4-CH₃—C₆H₄)— | -iso-C₃H₇ | m.p. = 61° C. |
| 75 | (3-NO₂-4-CH₃—C₆H₄)— | -sec-C₄H₉ | m.p. = 47° C. |
| 76 | (3-NO₂-4-CH₃—C₆H₄)— | —CH₃ | n_{D}²³ = 1.6000 |
| 77 | (3-NO₂-4-CH₃—C₆H₄)— | -iso-C₄H₉ | n_{D}²⁷ = 1.5236 |
| 78 | (3-NO₂-4-CH₃—C₆H₄)— | —C₂H₅ | n_{D}²⁷ = 1.5839 |
| 79 | 4-OCH₃—C₆H₄— | —C₆H₄-4-OCH₃ | m.p. = 84° C. |
| 80 | 4-C≡N—C₆H₄— | —C₆H₄-4-C≡N | m.p. = 146° C. |
| 81 | H₃C— | 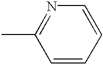 | m.p. = 57° C. |
| 82 | H₃C— | 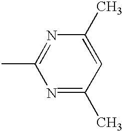 | m.p. = 108° C. |
| 83 | 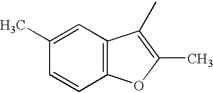 | 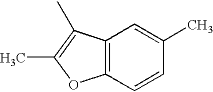 | m.p. = 142° C. |
| 84 | 4-F—C₆H₄— | —CH₆H₄-4-F | m.p. = 63° C. |
| 85 | 2-NO₂—C₆H₄— | —C₆H₄-2-NO₂ | m.p. = 196° C. |
| 86 | 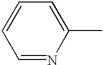 | -sec-C₄H₉ | n_{D}²² = 1.5200 |
| 87 | 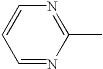 | -cyclo-C₆H₁₁ | ¹H NMR(CDCl₃): δ = 1.1-2.1, m, 10H; |
| 88 | 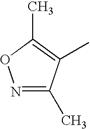 | -cyclo-C₆H₁₁ | N_{D}²⁶ = 1.5294 |
| 89 | 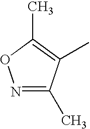 | —CH₂—(C₆H₄-4-Cl) | m.p. = 109° C. |
| 90 | 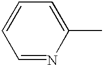 | -cyclo-C₆H₁₁ | N_{D}²⁴ = 1.5665 |
| 91 | 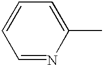 | -neo-C₅H₁₁ | N_{D}²⁴ = 1.5345 |
| 92 | 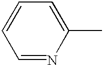 | —CH₂—(C₆H₄-4-Cl) | m.p. = 50° C. |
| 93 | 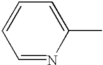 | —CH₂CH₂OH | N_{D}²³ = 1.5885 |

### USE EXAMPLE A

To demonstrate the activity against bacteria, the minimum inhibitory concentrations (MIC) of the agents according to the invention were determined:

The active compounds according to the invention were in each case added, in concentrations of from 0.1 mg/ml to 5000 mg/ml, to a chemically defined nutrient agar. After the agar has solidified, it was contaminated with pure cultures of the test organisms listed in Table 2. The MIC was determined after 3 days of incubation at 28° C. and 60 to 70% relative atmospheric humidity. MIC was the lowest concentration of active compound at which the microbial species used does not grow at all; it was stated in Table 2.

TABLE 2
Minimum inhibitory concentration (ppm) of compounds of the
formula (I) according to the invention

| Example | | |
|---|---|---|
| No. | Pseudomonas aeruginosa | Bacillus subtilis |
| 1 | <300 | <100 |
| 19 | <300 | <100 |
| 4 | <300 | <100 |
| 50 | <300 | <100 |
| 81 | <300 | <100 |
| 88 | <300 | <100 |

### USE EXAMPLE B

To demonstrate the activity against fungi, the minimum inhibitory concentrations (MIC) of agents according to the invention were determined:

The active compounds according to the invention were in each case added, in concentrations of from 0.1 mg/ml to 5000 mg/ml, to agar which was prepared using malt extract. After the agar has solidified, it was contaminated with pure cultures of the test organisms listed in Table 3. The MIC was determined after 2 weeks of incubation at 28° C. and 60 to 70% relative atmospheric humidity. MIC was the lowest concentration of active compound at which the microbial species used does not grow at all; it was stated in Table 3.

TABLE 3
Minimum Inhibitory concentrations (ppm) of compounds of
the formula (I) according to the invention

| Example | | Chaetomium | |
|---|---|---|---|
| No. | Penicillium brevicaule | globosum | Aspergillus niger |
| 12 | <200 | <300 | <400 |
| 23 | <200 | <300 | <400 |
| 32 | <200 | <300 | <400 |
| 67 | <200 | <300 | <400 |
| 76 | <200 | <300 | <400 |
| 87 | <200 | <300 | <400 |

### USE EXAMPLE C

To test dispersion paint coats for resistance to mould, the following procedure was adopted:

The paint to be tested was applied to both sides of a suitable base. To obtain results which were close to practice, some of the test specimens were leached out with running water (24 h, 20° C.) before the test for mould resistance; others were treated with a current of warm fresh air (7 days, 40° C.).

The samples prepared in this way were then placed on an agar nutrient medium, and both samples and nutrient medium were contaminated with fungal spores. After 2-3 weeks of storage (29±1° C., 80-90% rel. atmospheric humidity), the samples were compared.

The coating was considered to be permanently mould-resistant if the sample remains free from fungus or at most a slight infestation of the edge can be detected.

For the contamination, fungal spores of the following mould fungi were used, which were known as paint destroyers or were frequently encountered on coatings:

1. Alternaria tenius

2. Aspergillus flavus

3. Aspergillus niger

4. Aspergillus ustus

5. Cindosporum herbarum

6. Paecilomyces variotii

7. Penicillium citrium

8. Aureobasidium pullulans

9. Stachybotrys chartarum

Coatings according to recipe A were mould-resistant (even after leaching out and wind tunnel exposure) if they contain, for example, 1.5% (based on solids) of the compound of Example 23.

Recipe A: Exterior dispersion paint based on Acroal 290 D (styrene acrylate)

| | Parts by | |
|---|---|---|
| Trade name | weight | Chemical name |
| Bayer Titan RKB2 | 40 | Titanium dioxide |
| | 10 | Magnesium silicate, containing |
| Talkum V58 new | | water |
| Durcal 5 | 45 | Calcite CaCO₃ |
| Walsroder MC 3000 S 2% | 30 | Methylcellulose |
| H₂O | 6.5 | Distilled water |
| Calgon N 10% | 3 | Polyphosphate |
| Pigment distributor A 10% | 1 | Polyacrylic acid salt |
| Agitan 281, 1:1 in Texanol | 1 | |
| White spirit | 5 | Mixture of aliph. hydrocarbons |
| Butyl glycol acetate | 1.5 | Butyl glycol acetate |
| Acronal 290 D (binder) | 71 | Polyacrylic acid ester |
| Total | 219 | |

Solids content 135.5=61.6%.

### USE EXAMPLE D

To test the activity of compounds against wood-discoloring fungi, untreated pine wood was dipped into solutions of the compounds to be tested and then dried. The solvents were substances which have no fungicidal action, for example butanone, ethanol or dist. water.

For comparison, watered (24 h, 30° C.) and unwatered wood samples were placed onto an agar medium and contaminated with various mixed cultures. Following the inoculation with the mixed cultures, the samples were then stored separately at room temperature, and the extent of the infestation by the mixed cultures was assessed after a two-week incubation of the wood samples.

For contamination, fungal spores of the following fungi known to cause blue discoloration were used:

1. Aureobasidium pullulans

2. Sclerophoma pityophila

3. Trichoderma pseudokoningii

4. Gliocladium virens

5. Aspergillus niger

6. Ceratocystis pilifera

7. Cephaloascus fragans Hanawa

8. Phialophora fastigiata

9. Penicilium spec.

Sufficient protection against blue discoloration (even after watering) was given when the wood samples were dipped, for example, into a 1.5% strength solution (based on solids) of the compound of Example 26 in butanone.

Although the invention has been described in detail in the foregoing for the purpose of illustration, it was to be understood that such detail was solely for that purpose and that variations can be made therein by those skilled in the art without departing from the spirit and scope of the invention except as it may be limited by the claims.

What is claimed is:

1. A method for protecting an industrial material comprising treating the industrial material with a thiosulfonic acid ester microbiocide of the formula (I) 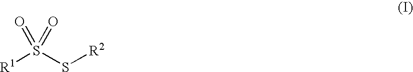
wherein R¹ and R² independently of one another represent in each case an optionally substituted alkyl, cycloalkyl, alkenyl, alkynyl, aryl or heterocyclyl.

2. The method of claim 1, wherein
R¹ and R² independently of one another represent an alkyl having from 1 to 10 carbon atoms, a cycloalkyl having 3 to 6 carbon atoms, an alkenyl having 2 to 10 carbon atoms or alkynyl having 2 to 10 carbon atoms, wherein R¹ and R² are optionally mono- or polysubstituted by identical or different substituents selected selected from the group consisting of halogen; hydroxyl; alkoxy having 1 to 6 carbon atoms; halogenoalkoxy having 1 to 6 carbon atoms and 1 to 9 identical or different halogen atoms; alkylthio having 1 to 6 carbon atoms; halogenoalkylthio having 1 to 6 carbon atoms and 1 to 9 identical or different halogen atoms; acyl having 1 to 6 carbon atoms; acyloxy having 1 to 6 carbon atoms; alkoxycarbonyl having 1 to 6 carbon atoms in the alkoxy moiety; amino which is optionally mono- or disubstituted by identical or different substituents selected from the group consisting of C₁-C₅-alkyl and aryl; optionally substituted phenoxy; optionally substituted aryl; optionally substituted pyridyl; optionally substituted pyridyloxy; nitro; cyano, or
R¹ and R² independently of one another represent aryl which is optionally mono- to pentasubstituted by identical or different substituents selected from the group consisting of halogen; alkyl having 1 to 10 carbon atoms; halogenoalkyl having 1 to 8 carbon atoms and 1 to 8 identical or different halogen atoms; alkoxy having 1 to 10 carbon atoms; halogenoalkoxy having 1 to 8 carbon atoms and 1 to 8 identical or different halogen atoms; halogenoalkylthio having 1 to 8 carbon atoms and 1 to 8 identical or different halogen atoms; amino; mono- or dialkylamino having in each case straight-chain or branched alkyl radicals having in each case 1 to 6 carbon atoms; nitro, cyano, or
R¹ and R² independently of one another represent heterocyclyl which is optionally mono- to pentasubstituted by identical or different substituents selected from the group consisting of halogen; alkyl having 1 to 10 carbon atoms; halogenoalkyl having 1 to 8 carbon atoms and 1 to 8 identical or different halogen atoms; alkoxy having 1 to 10 carbon atoms; halogenoalkoxy having 1 to 8 carbon atoms and 1 to 8 identical or different halogen atoms; halogenoalkylthio having 1 to 8 carbon atoms and 1 to 8 identical or different halogen atoms; amino; mono- or dialkylamino having in each case straight-chain or branched alkyl radicals having in each case 1 to 6 carbon atoms; nitro, cyano.

3. The method of claim 1, wherein the industrial material is selected from the group consisting of adhesives, sizes, paper, boards, leather, wood, paints, cooling lubricants and heat transfer fluids.

4. A method for controlling wood-discoloring or wood-destroying fungi on wood comprising treating the fungi with a thiosulfonic acid ester microbiocide of the formula (I) 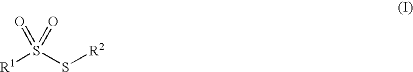
wherein R¹ and R² independently of one another represent in each case an optionally substituted alkyl, cycloalkyl, alkenyl, alkynyl, aryl or heterocyclyl; and protecting the industrial material.

5. The method of claim 4, wherein
R¹ and R² independently of one another represent an alkyl having from 1 to 10 carbon atoms, a cycloalkyl having 3 to 6 carbon atoms, an alkenyl having 2 to 10 carbon atoms or alkynyl having 2 to 10 carbon atoms, wherein R¹ and R² are optionally mono- or polysubstituted by identical or different substituents selected from the group consisting of halogen; hydroxyl; alkoxy having 1 to 6 carbon atoms; halogenoalkoxy having 1 to 6 carbon atoms and 1 to 9 identical or different halogen atoms; alkylthio having 1 to 6 carbon atoms; halogenoalkylthio having 1 to 6 carbon atoms and 1 to 9 identical or different halogen atoms; acyl having 1 to 6 carbon atoms; acyloxy having 1 to 6 carbon atoms; alkoxycarbonyl having 1 to 6 carbon atoms in the alkoxy moiety; amino which is optionally mono- or disubstituted by identical or different substituents selected from the group consisting of C₁-C₅-alkyl and aryl; optionally substituted phenoxy; optionally substituted aryl; optionally substituted pyridyl; optionally substituted pyridyloxy; nitro; cyano, or
R¹ and R² independently of one another represent aryl which is optionally mono- to pentasubstituted by identical or different substituents selected from the group consisting of halogen; alkyl having 1 to 10 carbon atoms; halogenoalkyl having 1 to 8 carbon atoms and 1 to 8 identical or different halogen atoms; alkoxy having 1 to 10 carbon atoms; halogenoalkoxy having 1 to 8 carbon atoms and 1 to 8 identical or different halogen atoms; halogenoalkylthio having 1 to 8 carbon atoms and 1 to 8 identical or different halogen atoms; amino; mono- or dialkylamino having in each case straight-chain or branched alkyl radicals having in each case 1 to 6 carbon atoms; nitro, cyano, or
R¹ and R² independently of one another represent heterocyclyl which is optionally mono- to pentasubstituted by identical or different substituents selected from the group consisting of halogen; alkyl having 1 to 10 carbon atoms; halogenoalkyl having 1 to 8 carbon atoms and 1 to 8 identical or different halogen atoms; alkoxy having 1 to 10 carbon atoms; halogenoalkoxy having 1 to 8 carbon atoms and 1 to 8 identical or different halogen atoms; halogenoalkylthio having 1 to 8 carbon atoms and 1 to 8 identical or different halogen atoms; amino; mono- or dialkylamino having in each case straight-chain or branched alkyl radicals having in each case 1 to 6 carbon atoms; nitro, cyano.

6. The method of claim 4, wherein the industrial material is selected from the group consisting of adhesives, sizes, paper, boards, leather, wood, paints, cooling lubricants and heat transfer fluids.

7. A microbicidal composition for the protection of a material, comprising
(a) a thiosulfonic acid ester microbiocide of the formula (I) 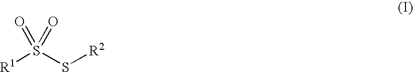
wherein R¹ and R² independently of one another represent in each case an optionally substituted alkyl, cycloalkyl, alkenyl, alkynyl, aryl or heterocyclyl; and protecting the industrial material.
(b) a solvent or a diluent;
(c) optionally a processing auxiliary
(d) optionally an active compound.

8. A process for preparing a microbicidal composition comprising
(a) a thiosulfonic acid ester microbiocide of the formula (I) 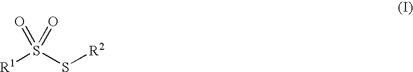
wherein R¹ and R² independently of one another represent in each case an optionally substituted alkyl, cycloalkyl, alkenyl, alkynyl, aryl or heterocyclyl;
(b) a solvent or a diluent;
(c) optionally a processing auxiliary, and
(d) optionally an active compound. comprising mixing a thiosulfonic acid ester microbiocide of the formula (I) with a solvent or a diluents and, optionally with a processing auxiliary an additional active compound, or a processing auxilary and an active compound.

9. An industrial material comprising at least one compound of the formula (I) according to claim 1.

10. A compound of the formula (I) 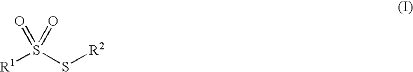
wherein
R¹ and R² independently of one another represent in each case an optionally substituted alkyl, cycloalkyl, alkenyl, alkynyl, aryl or heterocyclyl, except for
S-Methyl methanethiosulfonate
S-Ethyl ethanethiosulfonate
S-(1-Methyl)ethyl 2-methyl-ethanethiocarboxylate
S-Butyl butanethiosulfonate
S-(2-Methyl)propyl 2-methyl-propanethiocarboxylate
S-(1-Methyl)propyl 1-methyl-propanethiocarboxylate
S-(2,2-Dimethyl)propyl 2,2-dimethyl-propanethiocarboxylate
S-Methyl 4-toluenethiosulfonate
S-Methyl 4-chlorobenzenethiosulfonate
S-(1-Methyl)ethyl benzenethiosulfonate
S-(1,1-Dimethyl)ethyl benzenethiosulfonate
S-(2,2-Dimethyl)propyl benzenethiosulfonate
S-Butyl 4-toluenethiosulfonate
S-Cyclo-hexyl 4-toluenethiosulfonate
Ethyl 2-(4-chlorobenzene)-sulfonylsulfanyl-acetate
S-Cyclo-hexyl benzenethiosulfonate
Ethyl 3-benzenesulfonylsulfanyl-propionate
Ethyl 2-benzenesulfonylsulfanyl-acetate
S-(2-Phenylcarbamoyloxy)ethyl 4-toluenethiosulfonate
S-(2-Hydroxy)ethyl 4-toluenethiosulfonate
S-4-Tolyl 4-toluenethiosulfonate
S-(4-Methoxy)phenyl 4-methoxybenzenethiosulfonate
S-Methyl 2-pyridinethiosulfonate
S-(4-Cyano)phenyl 4-cyanobenzenethiosulfonate
S-(4-Fluoro)phenyl 4-fluorobenzenethiosulfonate
S-(2-Nitro)phenyl 2-nitrobenzenethiosulfonate

11. A process for preparing a compound of the formula (I) 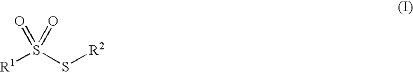
wherein
R¹ and R² independently of one another represent in each case an optionally substituted alkyl, cycloalkyl, alkenyl, alkynyl, aryl or heterocyclyl, except for
S-Methyl methanethiosulfonate
S-Ethyl ethanethiosulfonate
S-(1-Methyl)ethyl 2-methyl-ethanethiocarboxylate
S-Butyl butanethiosulfonate
S-(2-Methyl)propyl 2-methyl-propanethiocarboxylate
S-(1-Methyl)propyl 1-methyl-propanethiocarboxylate
S-(2,2-Dimethyl)propyl 2,2-dimethyl-propanethiocarboxylate
S-Methyl 4-toluenethiosulfonate
S-Methyl 4-chlorobenzenethiosulfonate
S-(1-Methyl)ethyl benzenethiosulfonate
S-(1,1-Dimethyl)ethyl benzenethiosulfonate
S-(2,2-Dimethyl)propyl benzenethiosulfonate
S-Butyl 4-toluenethiosulfonate
S-Cyclo-hexyl 4-toluenethiosulfonate
Ethyl 2-(4-chlorobenzene)-sulfonylsulfanyl-acetate
S-Cyclo-hexyl benzenethiosulfonate
Ethyl 3-benzenesulfonylsulfanyl-propionate
Ethyl 2-benzenesulfonylsulfanyl-acetate
S-(2-Phenylcarbamoyloxy)ethyl 4-toluenethiosulfonate
S-(2-Hydroxy)ethyl 4-toluenethiosulfonate
S-4-Tolyl 4-toluenethiosulfonate
S-(4-Methoxy)phenyl 4-methoxybenzenethiosulfonate
S-Methyl 2-pyridinethiosulfonate
S-(4-Cyano)phenyl 4-cyanobenzenethiosulfonate
S-(4-Fluoro)phenyl 4-fluorobenzenethiosulfonate
S-(2-Nitro)phenyl 2-nitrobenzenethiosulfonate
the process comprising reacting mercaptans of the formula (V) 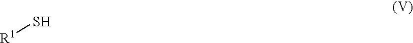
wherein R¹ represents optionally substituted alkyl, cycloalkyl, alkenyl, aryl or heterocyclyl,
with sulfinic acid sodium salts of the formula (IV) 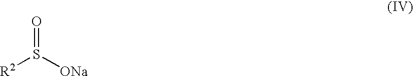
wherein R² represents optionally substituted alkyl, cycloalkyl, alkenyl, aryl or heterocyclyi,
optionally in the presence of a diluent and in the presence of a halogen

12. The process of claim 11, wherein the halogen is selected from the group consisting of bromine, chlorine and iodine.
